(19) United States
(12) Patent Application Publication
(10) Pub. No.: US 2019/0178837 A1
(43) Pub. Date: Jun. 13, 2019

(54) SENSING DEVICE AND METHOD IN DETECTING BINDING AFFINITY AND BINDING KINETICS BETWEEN MOLECULES

(71) Applicant: DEZHOU UNIVERSITY, Dezhou, Shandong (CN)

(72) Inventors: Shicai XU, Dezhou (CN); Jian ZHAN, Dezhou (CN); Jihua WANG, Dezhou (CN); Yaoqi ZHOU, Dezhou (CN)

(73) Assignee: DEZHOU UNIVERSITY, Dezhou, Shandong (CN)

(21) Appl. No.: 16/322,023

(22) PCT Filed: Nov. 30, 2016
(86) PCT No.: PCT/CN2016/107804

§ 371 Date: Jan. 30, 2019

(30) Foreign Application Priority Data
Sep. 20, 2016 (CN) 201610833102.0

(51) Int. Cl. G01N 27/414; G01N 33/557

(52) U.S. Cl. CPC G01N 27/4146; G01N 33/557

(57) ABSTRACT

A sensing device and method in detecting binding energy and binding kinetics between molecules; the sensing device has a sensor, a microfluidic chip and a measurement circuit. The sensor has multiple field effect transistors, and each field effect transistor adopts single-layer single crystal graphene as a conductive channel, thereby having extremely high sensitivity and stability; and the field effect transistors are arranged in an array and are provided with multichannel measurement circuits to detect the binding dynamics processes of different molecules or different copies of the same molecule in parallel so as to meet the high-throughput detection requirements. A compound that is non-covalently bound with the single-layer single crystal graphene as a medium to fix probe molecules on the surface of the conductive channel, thereby retaining the intrinsic structure of the graphene, and improving the signal-to-noise ratio and the sensitivity of the graphene field effect transistor.

### FIELD OF THE INVENTION

The present invention belongs to the technical field of sensing equipment and detection methods, and relates to a sensing device and method in detecting binding affinity and binding kinetics between molecules.

### BACKGROUND OF THE INVENTION

The detection of the binding affinity and the binding kinetics between molecules has an important application value in basic scientific research, screening and development of new drugs, disease diagnosis, and process control in food and pharmaceutical industries. Depending on whether the molecules to be tested are required a specific label, the detection methods can be classified into two types: depending on the label and label-free. The latter does not require the molecules to be labeled, but only depends on its physical properties such as molecular weight and charge amount for detection, thereby having the advantages of convenient operation, wide adaptability, and the like. The label-free detection of the binding affinity and the binding kinetics between the molecules is mainly a kind of sensors based on optical signal changes and based on the surface plasmon resonance (SPR) principle. Such sensors detect the quality change of the sensor surface before and after the combination of the molecules, and the sensitivity and accuracy thereof depend on the molecular weights of the molecules. For the molecules with large molecular weights, the kind of sensors has relatively high detection accuracy, but for the molecules with molecular weights less than 1000 Da, the sensitivity and accuracy are relatively low; in addition, the construction of this kind of sensors requires optical components such as a light source and a prism, which makes it difficult to achieve low cost, miniaturization and high-throughput detection. The personalization and precision requirements of medical treatment have challenged this kind of sensors, and the development of a novel sensor suitable for accurately detecting high and low molecular weights, having a low cost, a small size and portability, and being able to implement multichannel parallel high-throughput detection is an urgent need at present.

Graphene, a two-dimensional crystal composed of carbon atoms, can be used for detect certain molecules by detecting changes in the electrical resistance or conductance when used in a conductive channel of a field effect transistor (Schedin, F. et al. Nat. mater. 2007, 6, 652-655). Compared with SFR and other detection methods, the graphene sensor can make full use of the mature integrated manufacturing technology of the modern electronics industry to achieve low cost and miniaturization. This kind of sensors has stable physical and chemical properties, low detection limit, wide detection range, high detection accuracy, capability of realizing high integration and meeting the high-throughput detection requirements, and facilitating integration with tablet computers and smartphones, not only has wide application values in routine scientific research, engineering testing, disease diagnosis and other fields, and but also can be used in occasions with specific limitations on detection time, costs and equipment sizes, such as mobile care points, bedside diagnosis, ambulances and so on. Zuccaro, Laura, et al. ACS nano, 2015, 9(11), 11166-11176 reported a method for fabricating an enzyme affinity sensor by using a graphene field effect transistor, in which carboxylation is performed on the graphene via an electrochemical method, the topoisomerase is detected by using a fixed DNA probe, and an equilibrium constant of the interaction between the DNA and the topoisomerase is estimated to be 3.62±0.27 nM by using the electric field change caused by the topoisomerase, However, this method has many limitations: first, the graphene carboxylation process introduces many defects, thereby destroying the intrinsic structure of the graphene, and the sensitivity and the signal-to-noise ratio of the sensor are relatively poor; second, polycrystalline graphene is used for the graphene field effect transistor, the disordered distribution of crystal domains and defects cause poor performance uniformity of the graphene, the data measured by different devices are greatly different, and the accuracy of the results is poor; and third, the probe molecules are covalently bound with the graphene, so regeneration is difficult, and the sensor chip cannot be reused. Due to the restriction of factors such as sensitivity, signal-to-noise ratio, accuracy and non-regeneration, the existing sensors can only be used in limited analysis systems and can hardly be adapted to a broad-spectrum and standardized analysis method.

### SUMMARY OF THE INVENTION

The object of the present invention is to overcome the shortcomings of the existing technologies and provide a sensing device and method in detecting binding affinity and binding kinetics between molecules.

To achieve the above object, the technical solution of the present invention is as follows:

A sensor in detecting binding affinity and binding kinetics between molecules, wherein a field effect transistor in the sensor uses single-layer single crystal graphene as a conductive channel.

Preferably, multiple field effect transistors are arranged on the sensor, the multiple field effect transistors are arranged into a field effect transistor array, each field effect transistor uses the single-layer single crystal graphene as the conductive channel, and the field effect transistor array can perform parallel detection.

The field effect transistor array is a linear array, a rectangular array, an annular a a serpentine array or the like formed by the multiple field effect transistors.

Further preferably, the single crystal graphene in each field effect transistor is taken from the same large piece of single crystal graphene to improve the consistency of the performance of different graphene field effect transistor array points of the sensor.

Preferably, the sensor includes a compound A, and the compound A is non-covalently bound with the single crystal graphene.

Preferably, the compound A is a compound which can be both non-covalently bound with the single crystal graphene and covalently bound with probe molecules.

Further preferably, the sensor further includes probe molecules, and the probe molecules are covalently bound with the compound A.

Preferably, the field effect transistor is provided with single-layer single crystal graphene at the middle of an upper surface of an insulating substrate to form the conductive channel; and metal layers, insulating layers and microfluidic channel side walls are arranged above both sides of the single-layer single crystal graphene, so that the single-layer single crystal graphene, the insulating layers and the microfluidic channel side walls form a channel, wherein the metal layer on one side is the source of the field effect transistor, and the metal layer on the other side is the drain of the field effect transistor; and the gate of the field effect transistor is arranged above the single-layer single crystal graphene.

Preferably, multiple extraction electrodes are arranged on the sensor, and the extraction electrodes are conductors capable of leading the source or the drain of the field effect transistor to the outside of a microfluidic chip.

Preferably, the field effect transistor is a liquid gate type field effect transistor.

Further preferably, the insulating substrate is quartz, sapphire, silicon carbide or silicon with a thermal oxide layer.

Further preferably, the metal layer is a Cr/Au composite metal layer.

Still further preferably, the thickness of a Cr layer in the Cr/Au composite metal layer is 15-30 nm, and the thickness of an Au layer is 50-150 nm.

Further preferably, the insulating layer is prepared by atomic layer deposition or chemical vapor deposition.

Further preferably, the material of the insulating layer is Al₂O₃ or Si₃N₄.

Further preferably, the thickness of the insulating layer is 60-1.00 nm.

A sensing device in detecting binding affinity and binding kinetics between molecules includes the above sensor, a microfluidic chip and a measurement circuit.

A groove, a sample inlet, a sample outlet and a gate inlet are formed in the microfluidic chip, the sample inlet and the sample outlet respectively communicate both ends of the groove, the groove of the microfluidic chip and the upper surface of the sensor constitute a fluid channel, and the field effect transistor array in the sensor is located in the fluid channel, so that a fluid sample enters the fluid channel from the sample inlet, flows by the conductive channel of the field effect transistors and flows out from the sample outlet; and the gate of the sample outlet is aligned with the conductive channel through the gate inlet.

Preferably, multiple grooves, multiple sample inlets and multiple sample outlets are formed in the microfluidic chip, one sample inlet and one sample outlet respectively communicate both ends of one groove, the multiple grooves of the microfluidic chip and the upper surface of the sensor constitute multiple fluid channels, and the field effect transistors or the field effect transistor arrays in the sensor are distributed in the multiple fluid channels.

The measurement circuit includes one or more current sources, a microcontroller, and an A/D converter, and optionally includes one or more reference resistors; excitation current output by the current source passes through the drain and the source of the graphene field effect transistor through a drain interface and a source interface, and then passes through an optional reference resistor, and the A/D converter measures a voltage across the graphene field effect transistor and the optional reference resistor, and calculates the conductivity between the drain and the source of the graphene field effect transistor; and under the control of a computer, by monitoring the change of the conductivity between the drain and the source of the graphene field effect transistor with the gate voltage and the components of the liquid flowing by the graphene channel, the measurement circuit can measure a charge neutral point V_{cnp} of the graphene channel and a relative offset ΔV_{cnp} of the charge neutral point and output the same to the computer.

Preferably, the gate is a platinum wire, a nickel chrome wire or an Ag/AgCl electrode.

Preferably, the microfluidic chip is connected with the sensor by soldering, binding, fastening or clamping.

Preferably, the material of the microfluidic chip is glass, quartz, PMMA, PDMS, PEEK, PAEK, PC, PET, PS, PPS, PI, PSF, PVA or PVMK.

Preferably, the inner diameters of the sample inlet, the sample outlet and the gate inlet of the microfluidic chip are all less than 1 mm.

Preferably, the sensing device is connected with the computer via the measurement circuit. A specified voltage is output to the gate of the graphene field effect transistor under the control of the computer. The conductivity between the drain and the source of the graphene field effect transistor is measured simultaneously.

Further preferably, the measurement circuit is connected with the computer through a serial port, a parallel port, a USB interface or the Ethernet.

Further preferably, the measurement circuit includes one or more D/A converters connected to the gate interface, and the computer can control the D/A converter to output a specified voltage to the gate of the graphene field effect transistor,

Further preferably, the output range of the gate voltage of the measurement circuit is −10V to +10V, and the relative accuracy of the output is better than 1%.

Further preferably, the measurement circuit can be connected with multiple groups of sources, drains and gates of the sensing device at the same time. The purposes of multichannel parallel measurement and high-throughput detection can be achieved.

Further preferably, the relative accuracy of the measurement circuit for measuring the conductivity between the drain and the source is better than 1%, and the sampling speed is greater than 1 sps.

A detection system includes the above sensor and a compound A.

An application of the above sensor, the above sensing device or the above detection system in detecting binding energy and binding kinetics between molecules.

A method in detecting binding affinity and binding kinetics between molecules by using the above sensing device includes the following steps: firstly fixing probe molecules on the surface of the conductive channel of the single-layer single crystal graphene of the above sensor through the compound A, then injecting a solution to be tested into the sensing device through the microfluidic chip for detection, transmitting, by the sensing device, the detection data to the computer through the measurement circuit, and performing analysis and calculation according to detection data obtained by the computer.

Preferably, the compound A is bound with the surface of the single-layer single crystal graphene in a non-covalent manner, and the probe molecules are bound with the compound A in a covalent form to fix the probe molecules on the conductive channel of the sensor.

Preferably, the steps are as follows:

(1) functionalization of the sensor: injecting the solution of the compound A into the surface of the conductive channel of the sensor through the microfluidic chip, so that the compound A is non-covalently hound with the single crystal graphene, and then injecting the probe molecules into the surface of the conductive channel of the sensor through the microfluidic chip, so that the probe molecules are covalently bond with the compound A so as to fix the probe molecules on the conductive channel of the sensor through the compound A; or injecting the solution of the probe molecules that have been covalently bound with the compound A into the surface of the conductive channel of the sensor through the microfluidic chip, so that the probe molecules are fixed on the conductive channel of the sensor through the compound A;

(2) sample injection: injecting a buffer solution into the conductive channel of the sensor through the microfluidic chip until a detected baseline is stable; then injecting a solution to be tested into the conductive channel of the sensor through the microfluidic chip, so that molecules to be tested in the solution are bound with the probe molecules, and detecting parameters of the binding reaction; after the binding reaction reaches an equilibrium state, injecting the buffer solution into the conductive channel of the sensor through the microfluidic chip, so that the molecules to be tested are dissociated from the probe molecules, and detecting parameters of a dissociation process;

(3) data analysis: calculating to obtain a binding rate constant kₐ and a dissociation rate constant k_{d} via equations 1a-1b and parameters detected by the step (2), and obtaining an equilibrium constant K_{A} via a relational expression K_{A}=kₙ/k_{d};

or directly calculating the equilibrium constant K via an equation 2 and the parameters detected by the step (2);
the equations are

$$\begin{matrix}Δ {V}_{cnp} = Q \frac{{{k}_{a} [ B ]}_{ max} [ A ]}{{k}_{a} [ A ] + {k}_{d}} ( 1 - {e}^{- ( {k}_{a} [ A ] + {k}_{d} ) t )} ) , & ( 1 a ) \\ Δ {V}_{cnp} = Q \frac{{{k}_{a} [ B ]}_{ max} [ A ]}{{k}_{a} [ A ] + {k}_{d}} {e}^{- {k}_{d} t} , & ( 1 b ) \\ Δ {V}_{cnp} = {Q [ B ]}_{max} \frac{{K}_{A} [ A ]}{{K}_{A} [ A ] + 1} , & ( 2 )\end{matrix}$$

wherein, ΔV_{cnp} represents the relative offset of a graphene charge neutral point, [symbol]
represents a constant related to the charges of the molecules to be tested, the charge distribution of the molecules to be tested and a dielectric constant of the solution, kₐ represents the binding rate constant, k_{d} represents the dissociation rate constant, K_{A} represents the equilibrium constant, [A] represents the concentration of the molecules to be tested, and [B], represents the maximum density of the probe molecules.

Preferably, the buffer solution is a solution having a buffer function.

Further preferably, the buffer solution is phosphate (PBS) buffer solution, citrate (SSC) or tris(hydroxymethyl)aminomethane (Tris-HCl).

Further preferably, the compound A is 1-pyrenebutyric acid succinamide ester (PRASE), 1-pyrenebutyric acid, 4-(1-pyrenyl)-1-pyrenebutanol, 1-pyreneacetic acid, 1-pyrenecarboxylic acid or pyrene-1-boronic acid.

Preferably, the probe molecules are protein, DNA, RNA, small molecules or macromolecules.

Preferably, the molecules to be tested are protein, DNA, RNA, small molecules or macromolecules.

When the parameters of binding affinity and binding kinetics in a protein and ligand binding process are measured according to the present invention, the protein can be used as the probe molecules, and the ligand can be used as the molecules to be tested; or the ligand can be used as the probe molecules, and the protein can be used as the molecules to be tested. When the charge of the ligand is smaller, it is recommended to use the ligand as the probe molecules and to use the protein as the molecules to be tested; the probe molecules modified by each graphene field effect transistor array point can be the same and can also be different; and preferably, the molecular weight of the protein is from 5 kDa to 300 kDa, and the molecular weight of the ligand is from 0.1 kDa to 300 kDa.

When the parameters of binding affinity and binding kinetics between proteins are measured according to the present invention, the probe molecules are used a kind of protein, and the molecules to be tested are used as another kind of protein. The probe protein modified by each graphene field effect transistor array point can be the same and can also be different; preferably, the size of the probe protein is from 5 kDa to 300 kDa, and the size of the protein to be tested is from 5 kDa to 300 kDa; and the probe protein or the protein to be tested can be an antibody or an antigen.

When the parameters of binding affinity and binding kinetics in a DNA hybridization process are measured according to the present invention, the probe molecules are DNA, and the molecules to be tested are DNA or RNA. The DNA probe modified by each graphene field effect transistor array point can be the same and can also be different; preferably, the length of the DNA probe is 10 to 50 bases, the length of the DNA or RNA to be tested is 10 to 70 bases, and the probe DNA and the DNA or RNA to be tested can be completely complementary or partially complementary.

The covalent binding method of probe molecules and the compound A should be formulated based on the nature of the probe molecules and reactive groups. When the probe molecules have primary amino groups, a compound. A having an NHS ester group at the tail end can be selected to form a covalent amide bond connection between the probe molecules and the compound A in an aqueous solution with a pH of 7.2-8.5; and when the probe molecules have azide groups, the covalent connection with the compound A having an acetylene bond at the tail end can be formed by using a Huisgen azide-alkyne cycloaddition reaction.

When the parameters of binding affinity and binding kinetics in the DNA hybridization process are measured according to the present invention, preferably, a PBASE molecule having an NHS ester group at the tail end is used as the compound A that is non-covalently bound with the graphene; the solvent of the compound A solution in the step (1) is dimethylformamide (DMF), the concentration is 1-10 mM, the incubation temperature is the room temperature, and the incubation time is 1-3 h; the 5′ terminal aminated single-stranded DNA is used as the probe molecule; the concentration of the probe molecule solution is 50-100 μM, the solvent is a phosphate (PBS) buffer solution, the incubation temperature is the room temperature, and the incubation time is 1-3 h; the buffer solution in the step (2) is 0.005×-1×phosphate (PBS), or 0.005×-1×citrate (SSC); the injection rate of the DNA to be tested is 2-60 μl/min, which is constant; and the injection rate of the pure buffer solution in the dissociation process is also 2-60 μl/min, which is constant.

A method for restoring the detection capability of the above sensor includes the following steps: using a regeneration solution with weak strength to wash the molecules to be tested so as to restore the capability of the sensor to re-detect the molecules to be tested; or using a regeneration solution with great strength to wash the compound A, the probe molecules and the molecules to be tested so as to regenerate the sensor to re-detect the molecules to be tested.

Preferably, the regenerate solution with weak strength is 5-45 mM NaOH or 1-5 mM HCl, and the action time is 10-90 s for washing away the molecules to be tested.

Preferably, the regenerate solution with great strength is 50-100 mM NaOH or 5-10 mM HCl, and the action time is 120-300 s for washing away the compound A, the probe molecules and the molecules to be tested.

In the present invention, the graphene field effect transistor sensor adopts the single-layer single crystal graphene as the conductive channel of the field effect transistor, and has extremely high sensitivity and stability. The field effect transistors in the sensor are arranged in the array and are provided with multichannel measurement circuits to detect the binding dynamics processes of different biomolecules or different copies of the same biomolecule in parallel so as to meet the high-throughput detection requirements. The present invention adopts the compound A that is non-covalently bound with the graphene as a medium to fix the probe molecules, thereby retaining the intrinsic structure of the single-layer single crystal graphene, and improving the signal-to-noise ratio and the sensitivity of the graphene field effect transistor. By detecting the affinity and kinetics of DNA hybridization, the measurement error of device on the nanomolar DNA affinity and binding and dissociation rates is less than 10%, and the device can accurately identify the existence of single-site and multi-site mutation of the DNA sequence, the locations and the mutant nucleotide bases.

The present invention has the following beneficial effects

1. The device of the present invention adopts the single-layer single crystal graphene as the conductive channel of the field effect transistor, thereby improving the sensitivity and stability of the device and ensuring the reliability of the measurement result. When applied to the detection of DNA hybridization affinity, the measurement error of nanomolar DNA affinity is less than 10%.

2. The sensor used by the device of the present invention adopts the design of the graphene field effect transistor array and is provided with the multichannel measurement circuit to detect the binding energy and the dynamics processes of different molecules or different copies of the same molecule in parallel so as to meet the high-throughput detection requirements.

3. The device of the present invention uses the compound A that is non-covalently bound with the graphene as the medium to fix the probe molecules, thereby retaining the intrinsic structure of the single-layer single crystal graphene, and improving the signal-to-noise ratio and the sensitivity of the graphene. When applied to the detection of DNA hybridization affinity and the kinetic processes, the single-site mutation, multi-site mutation of the DNA sequence, and mutant nucleotide bases can be accurately distinguished.

4. The sensor used by the device of the present invention can be used repeatedly after reasonable regeneration, thereby reducing the cost of a single test.

5. The present invention provides the method for detecting the binding affinity and binding kinetics of molecules by using the above device, which has the characteristics of high analytical precision, high accuracy, reliable performance and reusability, and can be widely applied to multi-class molecular binding equilibrium constants and kinetic parameters, so that the method is expected to become a standardized analysis method and has an important application value in basic research of life sciences, screening and development of new drugs, disease diagnosis, process control in food and pharmaceutical industries.

### BRIEF DESCRIPTION OF THE DRAWINGS

FIG. 1 is a structural schematic diagram of a sensing device of the present invention.

FIG. 2 is an optical micrograph of a sensor (a grapheme field effect transistor array) of a sensor of the present invention.

FIG. 3 is a schematic diagram of a section of a single graphene field effect transistor of the present invention.

FIG. 4 is a schematic diagram of a measurement circuit of the present invention.

FIG. 5 is a photo of assembly between a sensor and a microfluidic chip in an embodiment 1.

FIG. 6 is a scanning electron microscope (SEM) image of single-layer single crystal graphene used in the embodiment 1.

FIG. 7 is a schematic diagram of fixing probe molecules on the surface of graphene and binding between the molecules to be tested and the probe molecules in an embodiment 2.

FIG. 8 is a kinetic sensing diagram of a DNA hybridization process measured in the embodiment 2.

FIG. 9 is a relationship graph between a DNA hybridization response equilibrium value and DNA concentration measured in the embodiment 2.

FIG. 10 is a kinetic sensing contrast diagram of complete match DNA hybridization and single site mismatch hybridization measured in an embodiment 3.

FIG. 11 is a relationship graph between a response equilibrium value and DNA concentration corresponding to the complete match DNA hybridization and single site mismatch hybridization measured in the embodiment 3.

FIG. 12 is a kinetic sensing diagram of DNA different site mismatch DNA hybridization process measured in an embodiment 4.

FIG. 13 is a kinetic sensing diagram of different types of mutation measured in an embodiment 5.

FIG. 14 is a result of 5 times of detection capability restoration (regeneration) and detection results of DNA T20 with different concentrations after regeneration using the sensing device in an embodiment 6.

### REFERENCE SIGNS

1, microfluidic chip, 2, sensor, 3, source extraction electrode, 4, drain extraction electrode, 5, gate inlet, 6, sample inlet, 7, groove, 8, sample outlet, 9, graphene field effect transistor array, 10, source, 11, drain, 12, single-layer single crystal graphene, 13, insulating layer, 14, microfluidic channel side wall, 15, gate, 16, solution to be tested, and 17, insulating substrate.

### DETAILED DESCRIPTION OF THE EMBODIMENTS

The present invention will be farther described below in combination with the drawings and embodiments.

### Embodiment 1

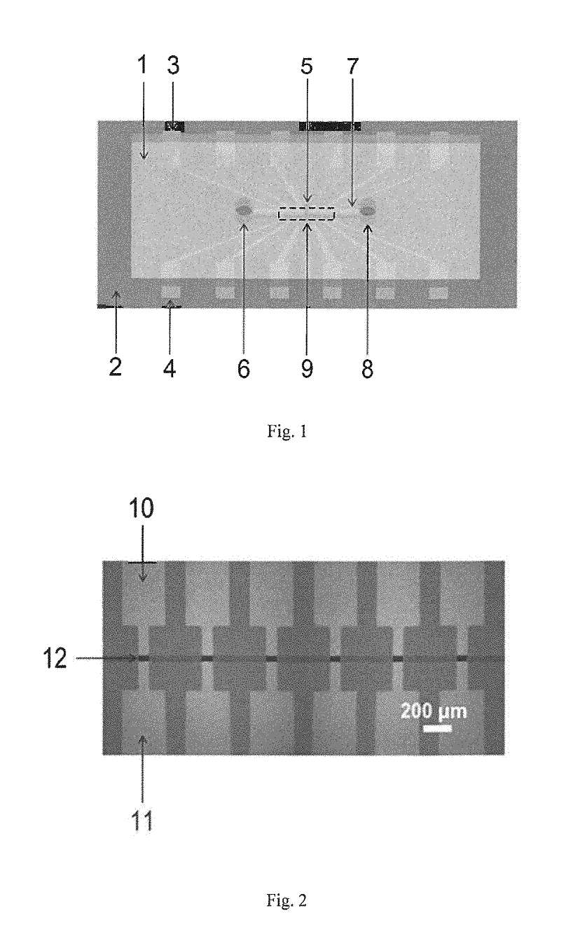

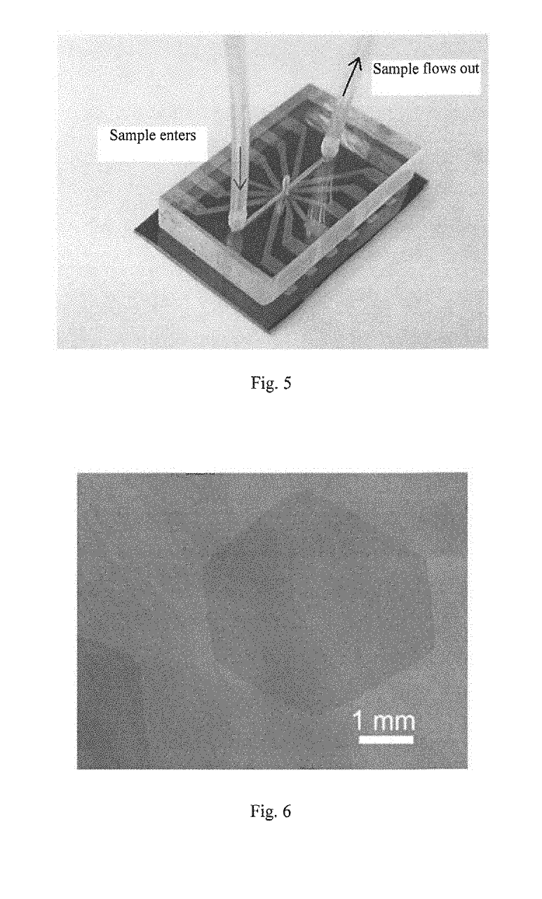

A sensing device in detecting binding affinity and binding kinetics between molecules, as shown in FIGS. 1 and 5, includes a sensor 2 and a microfluidic chip 1; and

as shown in FIG. 2, multiple field effect transistors are arranged on the sensor 2, the multiple field effect transistors are arranged into a field effect transistor array 9, each field effect transistor is provided with a conductive channel composed of single-layer single crystal graphene 12, and all field effect transistor arrays form multiple parallel detection channels. The used single-layer single crystal grapheme is as shown in FIG. 6.

A groove 7, a sample inlet 6, a sample outlet 8 and a gate inlet 5 are formed in the microfluidic chip 1, the sample inlet 6, the sample outlet 8 and the gate inlet 5 are formed in the upper side of the microfluidic chip 1, and the sample inlet 6 and the sample outlet 8 respectively communicate both ends of the groove 7; and

the sensor 2 is installed on one side of the microfluidic chip 1 with the groove 7, the groove 7 and the sensor 2 form a fluid channel, the field effect transistor array 9 is located in the fluid channel, so that a solution to be tested 16 enters the fluid channel from the sample inlet 6, flows by the fluid channel and flows out from the sample outlet 8, and the solution to be tested 16 is in contact with the single-layer single crystal graphene 12 serving as the conductive channel of the field effect transistor while flowing by the fluid channel; and the gate of the field effect transistor is aligned with the single-layer single crystal graphene 12 serving as the conductive channel of the field effect transistor through the gate inlet 5.

Multiple extraction electrodes are arranged on the sensor 2 and include source extraction electrodes 3 and drain extraction electrodes 4; and the extraction electrodes are conductors capable of leading the source or the drain of the field effect transistor to the outside of the microfluidic chip, so that a measurement circuit can be connected to the source or the drain.

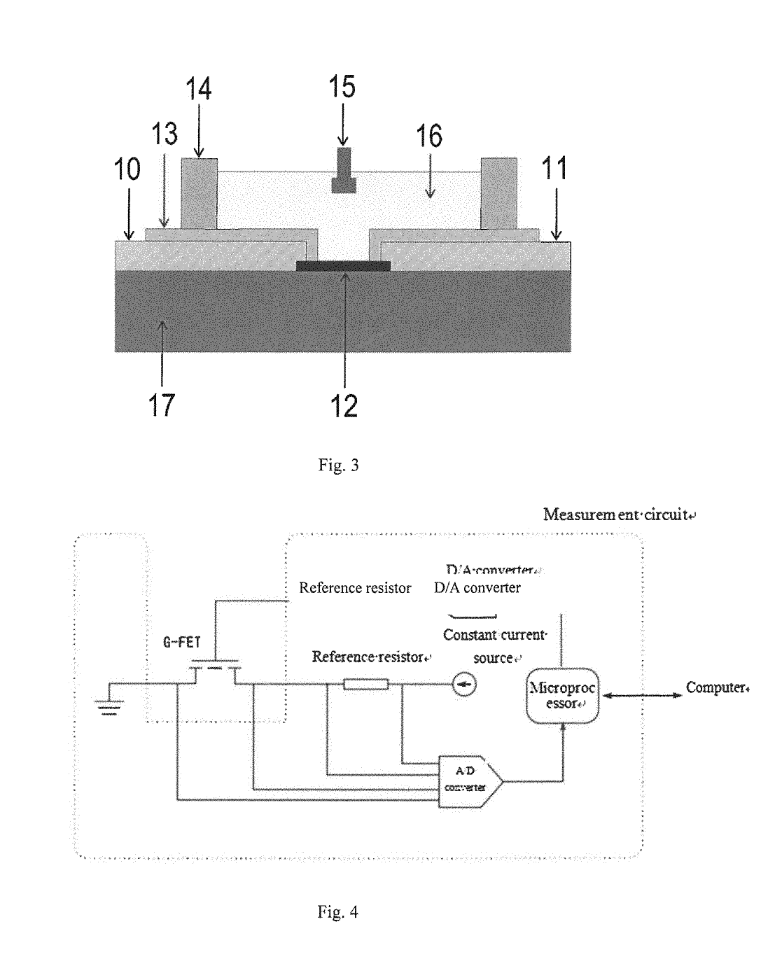

As shown in FIGS. 2 to 3, the field effect transistor in the sensor 2 is provided with the single-layer single crystal graphene 12 at the middle of an upper surface of an insulating substrate 17 to serve as the conductive Channel; and metal layers, insulating layers 13 and microfluidic channel side walls 14 are arranged above both sides of the single-layer single crystal graphene 12, the gate 15 of the field effect transistor is arranged above the single-layer single crystal graphene 12, wherein the metal layer on one side is the source 10 of the field effect transistor, and the metal layer on the other side is the drain 11 of the field effect transistor.

In the sensor 2, the metal layers on both sides of the single-layer single crystal graphene 12 are exposed on one side away from the single-layer single crystal graphene.

The sensor 2 adopts Si with a 300 nm thermal oxide layer as the insulating substrate 17; the source 10 and the drain 11 are deposited by 20 nm Cr and 100 nm Au composite metal layers through a magnetron sputtering method and are respectively extracted by the source extraction electrodes 3 and the drain extraction electrodes 4 to be connected with the measurement circuit; the field effect transistor adopts a liquid top gate structure, a platinum wire is used as the gate 15, and the gate enters the groove 7 from the gate inlet 5 so as to provide a gate voltage for the field effect transistor; a 80 nm Si₃N₄ insulating layer is plated on the upper surface (excluding the area where the single-layer single crystal graphene 12 and an electrode extraction end are located) of the field effect transistor as a top insulating layer 13, in order to eliminate the parasitic current among the gate, the source and the drain. The microfluidic chip 1 made of a PMMA material is fixed on the top of the sensor 2 by binding, the single-layer single crystal graphene 12 is located in the fluid channel, the sample inlet 6 and the sample outlet 8 are located at both ends of the groove 7, and a sample solution 16 flows in from the sample inlet 6 and flow out from the sample outlet 8. The gate 15 of the graphene field effect transistor array 9 is connected with a gate voltage output end of the measurement circuit, the measurement circuit is as shown in FIG. 4, and the drain 11 thereof is connected with the drain end of the measurement circuit through the drain extraction electrode 4, and the source 10 is connected with a source end of the measurement circuit through the source extraction electrode 3. The measurement circuit is connected with a computer, can obtain measurement data through software and can perform data analysis.

### Embodiment 2

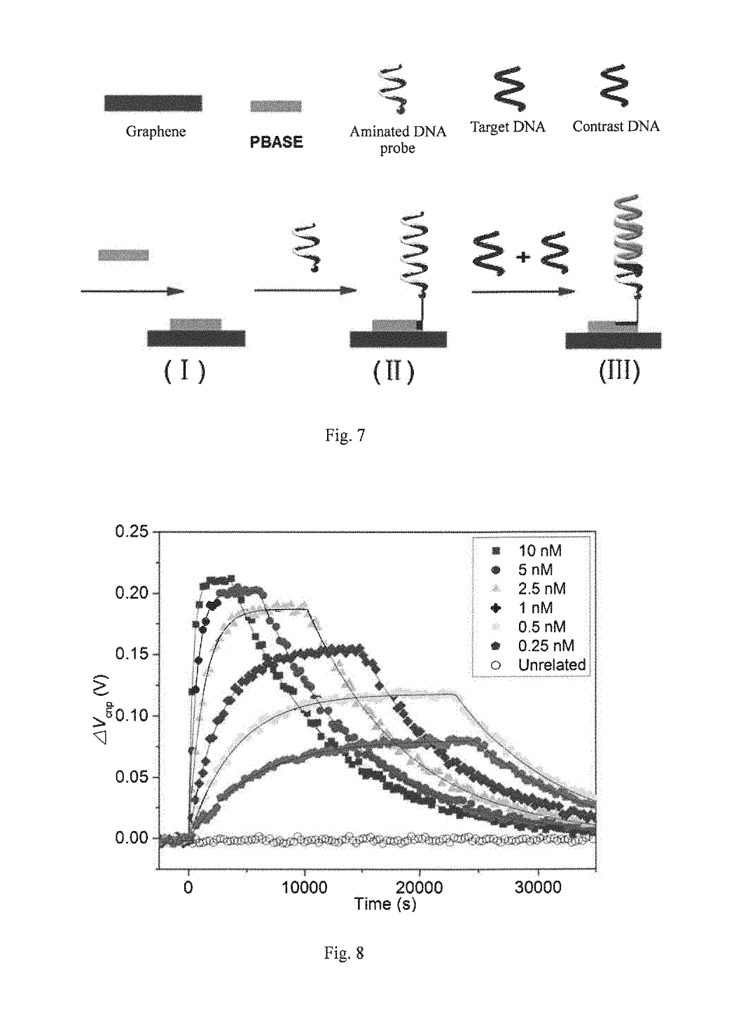

The DNA hybridization affinity of using the device of the embodiment 1. The process of fixing probe molecules to the surface of the graphene and binding molecules to be tested with the probe molecules is shown in FIG. 7.

(1) a dimethylformamide (DMF) solution of 10 mM 1-pyrenebutyric acid succinamide ester (PBASE) is injected into the surface of the graphene single crystal by using an injection pump through the microfluidic chip, pure DMF is injected to wash away the excess PBASE after incubation at the room temperature for 1 h; 100 mM 5′ terminal aminated single-stranded DNA (sequence: H₂N—(CH₂)₆-5′-GAGTTGCTACAGACCTTCGT-3′, serial number: P20) aqueous solution is injected into the surface of the graphene, incubation is performed at the room temperature for 6 h to fix a DNA probe P20 to the surface of the graphene single crystal;

(2) the DNA to be tested (sequence: 3′-CTCAACGATGTCTGGAAGCA-5′, serial number: T 20) is added into a 0.01×PBS buffer solution to prepare a sample solution group to be tested (concentration: 0.25, 0.5, 1, 2.5, 5, 10 nM), and an unrelated sequence DNA is set as a control experiment (sequence: 3′-ACATGTAGGTTTGATATGAT-5′, serial number: U20); the sample solution is injected into the functionalized surface of the graphene by using the injection pump through the microfluidic chip, a constant injection rate of 60 μl/min is kept, and the hybridization reaction process is monitored via the measurement circuit; after the hybridization reaction reaches equilibrium, the sample solution containing the DNA to be tested is switched to a pure buffer solution, which is injected by the injection pump at the constant injection rate of 60 μl/min, and a dissociation process of double-stranded DNA is monitored; and

(3) a kinetic constant and an equilibrium constant are obtained by a real-time kinetic process: a binding rate constant kₐ and a dissociation rate constant k_{d} are obtained by fitting an equation (1), and an equilibrium constant K_{A} is obtained by a relational expression K_{A}=kₐ/k_{d}.

The fitting results are shown in the following table:

TABLE 1
P20-T20 hybridization kinetic parameters and equilibrium constants
obtained by fitting different graphene field effect transistors

| | G-FET 1 | G-FET 2 | G-FET 3 | G-FET 4 | G-FET 5 | G-FET 6 |
|---|---|---|---|---|---|---|
| kₐ (× 10⁵M⁻¹ s⁻¹) | 2.61 (0.11)^{c} | 2.68 (0.12) | 2.36 (0.13) | 2.73 (0.09) | 2.38 (0.20) | 2.70 (0.18) |
| k_{d} (×10⁻⁴ s⁻¹⁾ | 1.08 (0.07) | 1.13 (0.04) | 1.02 (0.06) | 1.23 (0.10) | 1.10 (0.08) | 1.15 (0.07) |
| K_{A} (×10⁹M⁻¹)^{a} | 2.35 | 2.37 | 2.31 | 2.22 | 2.16 | 2.42 |
| K_{A} (×10⁹M⁻¹)^{b} | 2.37 | 2.30 | 2.26 | 2.23 | 2.11 | 2.39 |

^{a}is obtained by the relational expression K_{A} = kₐ/k_{d};
^{b}is obtained by fitting a relation curve between equilibrium response values and the concentration of the DNA to be tested;
the value of ^{c} is derived from the mean of the fitting results of the independent measurements of the six channels, standard deviations of the detection of T20 with different concentrations obtained after the regeneration of the sensing device are shown in the brackets, and the rest is similar.

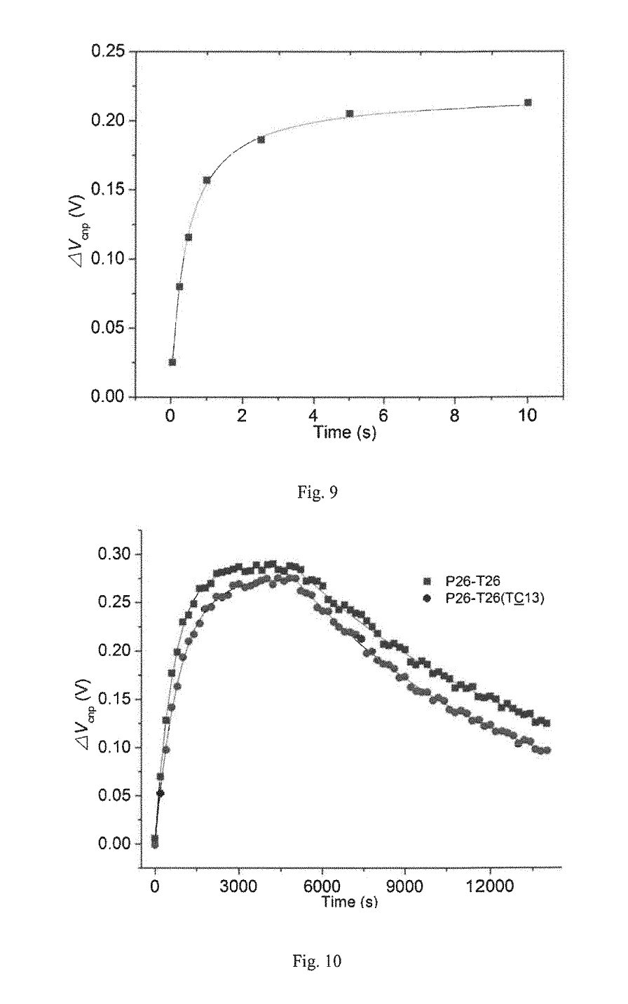

The results measured in the present embodiment are shown in FIGS. 8 to 9.

### Embodiment 3

The difference between complete match I)NA hybridization and single site mismatch hybridization is compared by using the device of the embodiment 1.

As described in the embodiment 2, the difference lies in that:

In step (1), the concentration of the PRASE is 5 mM, and the 5′ terminal aminated single-stranded DNA (sequence: H2N—(CH2)6-5′-ACCAGGCGGCCGCACACGTCCTCCAT-3′; serial number: P26);

In the step (2), the DNA to be tested are complete match DNA (sequence: 3′-TGGTCCGCCGGCGTGCAGGAGGTA-5′, serial number: T26) and single site mismatch DNA (sequence: 3′-TGGTCCGCCGGCGCGTGCAGGAGGTA-5′, serial number: T26 (TC13); the concentrations of the two DNA sample solutions to be tested are 5 nM;

Step (3) is the same as the embodiment 2, and the fitting results are shown in the following table:

TABLE 2
Kinetic parameters and equilibrium constants of
hybridization of P26-T26 and P26-T26 (TC13)

| | kₐ (×10⁵ M⁻¹ s⁻¹) | k_{d} (×10⁻⁴ s⁻¹) | K_{A} (×10⁹ M⁻¹) |
|---|---|---|---|
| P26-T 26 | 2.87 (0.18) | 9.26 (0.13) | 3.10 (0.21) |
| P26-T26 (TC13) | 2.10 (0.21) | 1.17 (0.10) | 1.80 (0.18) |

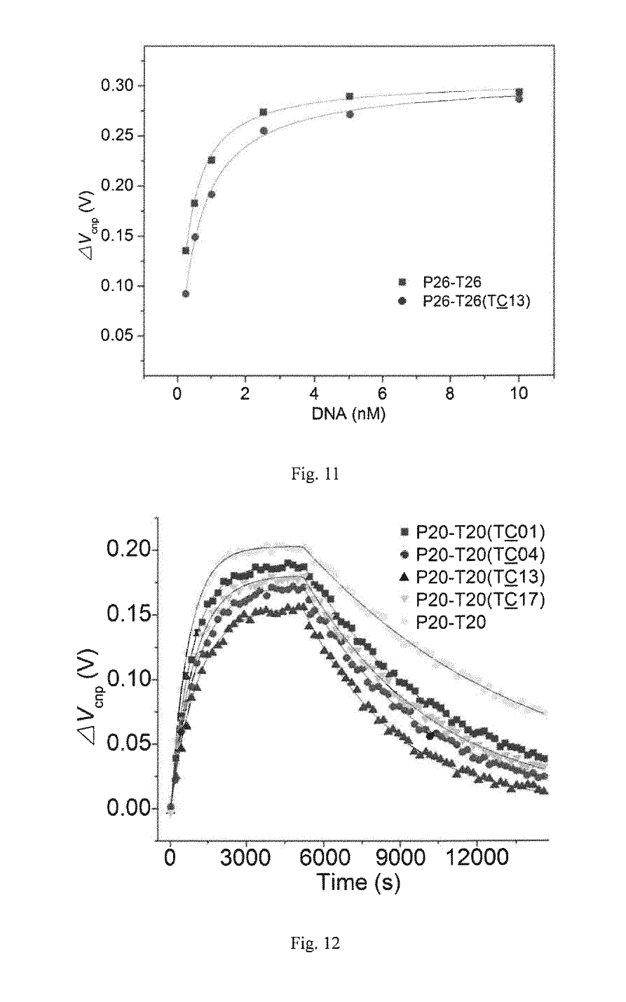

The results measured in the present embodiment are shown FIGS. 10 to 11.

### Embodiment 4

The difference of DNA different site mismatch DNA hybridization is compared by using the device of the embodiment 1.

As described in the embodiment 2, the difference lies in that:

In step (1), the concentration of the PBASE is 8 mM, and 50 mM probe DNA P20 aqueous solution is injected into the surface of graphene, and incubation is performed at the room temperature for 8 h;

In step (2), the DNA to be tested are complete match DNA (T20) and four different single site mismatch DNAs (sequence: 3′-CTCAACGATGTCTGGAAGCC-5′, serial number: T20 (TC01); sequence: 3′-CTCAACGAMTCTGGACGCA-5′, serial number: T20 (TC04); sequence: 3′-CTCAACGTGTCTGGAAGCA-5′, serial number: T20 (TC13); sequence: 3′-CTCCACGATGTCTGGAAGCA-5′, serial number: T20 (TC17)); the concentrations of the five DNA sample solutions to be tested are 5 nM; and 5 kinds of The concentration of the DNA sample solution was 5 nM;

Step (3) is the same as the embodiment and the fitting results are shown in the following table:

TABLE 3
Kinetic parameters and equilibrium constants of hybridization
of P20 with complete match and different site mismatch

| | kₐ (×10⁵ M⁻¹ s⁻¹) | k_{d} (×10⁻⁴ s⁻¹) | K_{A} (×10⁹ M⁻¹) |
|---|---|---|---|
| P20-T20 | 2.62 (0.14) | 1.09 (0.07) | 2.40 (0.15) |
| P20-T20 (TC01) | 2.15 (0.15) | 1.78 (0.08) | 1.21 (0.16) |
| P20-T20 (TC04) | 1.61 (0.12) | 2.19 (0.04) | 0.74 (0.14) |
| P20-T20 (TC13) | 1.39 (0.14) | 2.83 (0.07) | 0.49 (0.13) |
| P20-T20 (TC17) | 1.82 (0.16) | 1.98 (0.09) | 0.92 (0.17) |

The kinetic sensing diagram of the DNA different site mismatch DNA hybridization process measured in the present embodiment is shown in FIG. 12.

### Embodiment 5

Different types of mutation are distinguished by using the device of the embodiment 1.

As described in the embodiment 2, the difference lies in that:

In step (1), the concentration of the PBASE is 5 mM, and the 5′-terminally aminated probe DNA (sequence: H2N—(CH2)6-5′-TTTTTTCGGCCGCACACGTCC-3′; serial number: P15);

In the step (2), there are two kinds of DNA to be tested, in one DNA to be tested, T at the 13^{th} site of the 5′ end mutates to C (sequence: 3′-TGGTCCGCCGGCGTGTGCAGGAGGTA-5′, serial number: T26 (TC13)), in the other DNA to be tested, the T at the 13^{th} site of the 5′ end mutates to G (sequence: 3′-TGGTCCGCCGGCGGGTGCAGGAGGTA-5′, serial number: T26 (TG13)); the concentrations of the two DNA sample solutions to be tested are 5 nM;

Step (3) is the same as the embodiment 2, and the fitting results are shown in the following table:

TABLE 2
Kinetic parameters and equilibrium constants of
P15-T26 (TG13)T26, P15-T26 (TC13) hybridization

| | kₐ (×10⁵ M⁻¹ s⁻¹) | k_{d} (×10⁻⁴ s⁻¹) | K_{A} (×10⁹ M⁻¹) |
|---|---|---|---|
| P26-T26 (TG13) | 1.11 | 5.07 | 0.22 |
| P26-T26 (TC13) | 1.04 | 8.53 | 0.12 |

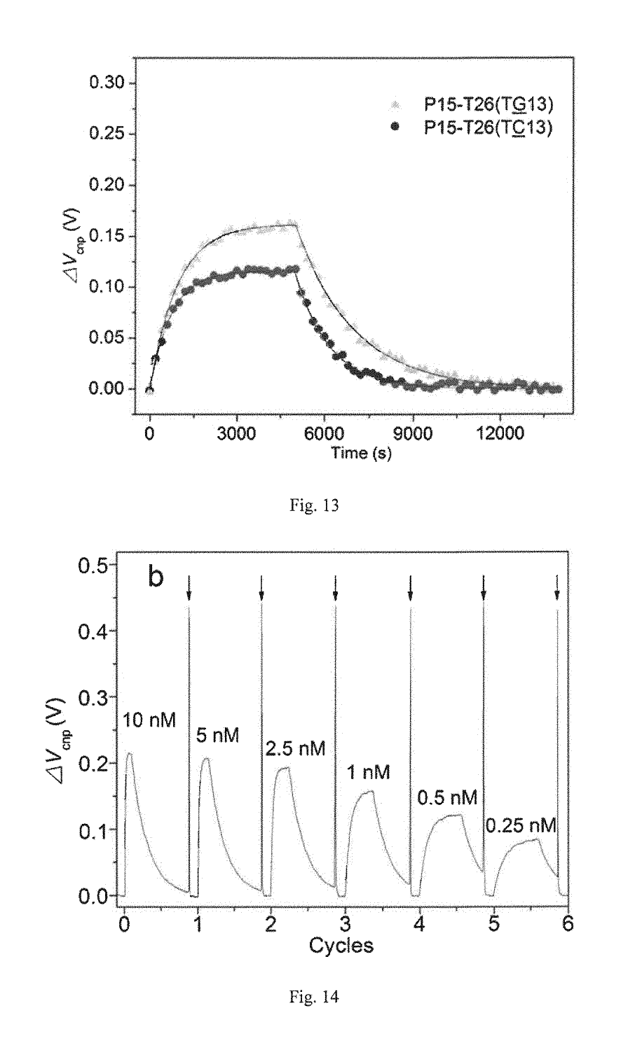

The kinetic sensing diagram of different types of mutations measured in the present embodiment is shown in FIG. 13.

### Embodiment 6

The detection capability of the sensing device of the embodiment 1 is restored by regeneration.

As described in the embodiment 2, the difference lies in that, after the completion of the embodiment 2, the detection capability of the sensing device is restored, including the following steps:

(1) using an injection pump to inject 15 mM NaOH into the surface of the functionalized graphene through the microfluidic chip, maintaining a constant injection rate of 30 μl/min and an injection time of 60 s, and unwinding P20-T20; and

(2) using the injection pump to inject 0.01×PBS buffer solution into the surface of the functionalized graphene through the microfluidic chip to wash away the DNA T20 to be tested and to restore the specific binding capability of the sensing device P20, wherein the detection result is as shown in FIG. 14.

### Embodiment 7

The detection capability of the sensing device of the embodiment 1 is restored by regeneration.

As described in the embodiment 2, the difference lies in that, after the completion of the embodiment 2, the capability of re-detecting new molecules of the sensing device is restored, including the following steps:

(1) using the injection pump to inject 80 mM NaOH into the surface of the functionalized graphene through the microfluidic chip, maintaining a constant injection rate of 30 μl/min and an injection time of 150 s, and eluting the PBASE, the DNA to be tested T20 and the probe DNA P20 from the surface of the graphene;

(2) using the injection pump to inject 0.01×PBS buffer solution into the surface of the functionalized graphene through the microfluidic chip to wash away the PBASE, the DNA to be tested T20 and the probe DNA P20 from the surface of the graphene at the same time; and

(3) repeating the step (1) in the embodiment 2, linking the probe DNA P20 to the surface of the graphene again to restore the specific binding ability of the sensing device P20.

Although the specific embodiments of the present invention have been described in combination with the drawings, the protection scope of the present invention is not limited thereto, and those skilled in the art to which the present invention belongs should understand that, various modifications or variations, made by those skilled in the art on the basis of the technical solutions of the present invention without any creative effect, still fall within the protection scope of the present invention.

1. A sensor in detecting binding affinity and binding kinetics between molecules, wherein a field effect transistor in the sensor uses single-layer single crystal graphene as a conductive channel.

2. The sensor in detecting binding affinity and binding kinetics between molecules according to claim 1, wherein multiple field effect transistors are arranged on the sensor, the multiple field effect transistors are arranged into a field effect transistor array, each field effect transistor uses the single-layer single crystal graphene as the conductive channel, and the field effect transistor array can perform parallel detection.

3. The sensor in detecting binding affinity and binding kinetics between molecules according to claim 1, comprising a compound A, wherein the compound A is non-covalently bound with the single crystal graphene.

4. The sensor in detecting binding affinity and binding kinetics between molecules according to claim 3, further comprising probe molecules, wherein the probe molecules are covalently bound with the compound A.

5. A sensing device in detecting binding affinity and binding kinetics between molecules, comprising a sensor, a microfluidic chip and a measurement circuit, wherein the sensor is the sensor according to claim 1.

6. A detection system comprising the sensor according to claim 1 and a compound A.

7. A method comprising detecting binding affinity and binding kinetics between molecules with the sensor according to claim 1.

8. A method in detecting binding affinity and binding kinetics between molecules, comprising the following steps: firstly fixing probe molecules on the surface of the conductive channel of the single-layer single crystal graphene of the sensor according to claim 1 through the compound A, then injecting a solution to be tested into the sensing device through the microfluidic chip for detection, transmitting, by the sensing device, the detection data to the computer through the measurement circuit, and performing analysis and calculation according to detection data obtained by the computer.

9. The method according to claim 8, wherein the steps are as follows:
(1) functionalization of the sensor: injecting the solution of the compound A into the surface of the conductive channel of the sensor through the microfluidic chip, so that the compound A is non-covalently bound with the single crystal graphene, and then injecting the probe molecules into the surface of the conductive channel of the sensor through the microfluidic chip, so that the probe molecules are covalently bond with the compound A so as to fix the probe molecules on the conductive channel of the sensor through the compound A; or injecting the solution of the probe molecules that have been covalently bound with the compound A into the surface of the conductive channel of the sensor through the microfluidic chip, so that the probe molecules are fixed on the conductive channel of the sensor through the compound A;
(2) sample injection: injecting a buffer solution into the conductive channel of the sensor through the microfluidic chip until a detected baseline is stable; then injecting a solution to be tested into the conductive channel of the sensor through the microfluidic chip, so that molecules to be tested in the solution to be tested are bound with the probe molecules, and detecting parameters of the binding reaction; after the binding reaction reaches an equilibrium state, injecting the buffer solution into the conductive channel of the sensor through the microfluidic chip, so that the molecules to be tested are dissociated from the probe molecules, and detecting parameters of a dissociation process;
(3) data analysis: calculating to obtain a binding rate constant kₐ and a dissociation rate constant k_{d} via equations 1a-1b and parameters detected by the step (2), and obtaining an equilibrium constant K_{A} via a relational expression K_{A}=kₐ/k_{d};
or directly calculating the equilibrium constant K_{A} via an equation 2 and the parameters detected by the step (2);
the equations are
$$\begin{matrix}Δ {V}_{cnp} = Q \frac{{{k}_{a} [ B ]}_{ max} [ A ]}{{k}_{a} [ A ] + {k}_{d}} ( 1 - {e}^{- ( {k}_{a} [ A ] + {k}_{d} ) t )} ) , & ( 1 a ) \\ Δ {V}_{cnp} = Q \frac{{{k}_{a} [ B ]}_{ max} [ A ]}{{k}_{a} [ A ] + {k}_{d}} {e}^{- {k}_{d} t} , & ( 1 b ) \\ Δ {V}_{cnp} = {Q [ B ]}_{max} \frac{{K}_{A} [ A ]}{{K}_{A} [ A ] + 1} , & ( 2 )\end{matrix}$$
wherein, ΔV_{cnp} represents the relative offset of a graphene charge neutral point, [symbol]
represents a constant related to the charges of the molecules to be tested, the charge distribution of the molecules to be tested and a dielectric constant of the solution, kₐ represents the binding rate constant, k_{d} represents the dissociation rate constant, K_{A} represents the equilibrium constant, [A] represents the concentration of the molecules to be tested, and [B]ₘₐₓ represents the maximum density of the probe molecules.

10. A method for restoring the detection capability of the sensor according to claim 1, comprising the following steps: using an regeneration solution with weak strength to wash the molecules to be tested so as to restore the capability of the sensor to re-detect the molecules to be tested; or using an regeneration solution with great strength to wash the compound A, the probe molecules and the molecules to be tested so as to regenerate the capability of the sensor to re-detect the molecules to be tested.
